(19) United States
(12) Patent Application Publication
(10) Pub. No.: US 2012/0240246 A1
(43) Pub. Date: Sep. 20, 2012

(54) Variant Calpastatins and Variant Calpains for Modulating the Activity or Stability of Calpain

(75) Inventor: Tudor Moldoveanu, Memphis, TN (US)

(73) Assignee: St. Jude Children's Research Hospital, Memphis, TN (US)

(21) Appl. No.: 13/508,995

(22) PCT Filed: Nov. 17, 2010
(86) PCT No.: PCT/US10/56928

§ 371 Date: May 10, 2012

Related U.S. Application Data
(60) Provisional application No. 61/261,802, filed on Nov. 17, 2009.

(51) Int. Cl. A23L 1/318; C07K 19/00; C12N 15/12; C12N 15/70; A61K 38/57; C12N 9/96; C12N 9/99; C12N 15/57; C12N 1/21; C07K 14/81; A01K 67/00

(52) U.S. Cl. 800/13; 530/350; 536/23.5; 435/320.1; 435/188; 435/184; 536/23.2; 435/252.33; 426/281; 514/20.2

(57) ABSTRACT

The present invention features stabilized/destabilized variant calpastatin proteins and peptides that modulate the stability/activity of calpain for use in analyzing the pathophysiology of diseases associated with calpain activity, facilitating muscle growth and in improving meat tenderization.

### INTRODUCTION

This application claims benefit of priority to U.S. Provisional Application Ser. No. 61/261,802, filed Nov. 17, 2009, the content of which is incorporated herein by reference in its entirety.

### BACKGROUND OF THE INVENTION

The calpains are the only known mammalian cysteine proteases directly activated by calcium. There are 15 members in mammals with the most abundant and ubiquitously expressed isoforms being calpain I or μ-calpain and calpain II or m-calpain. The former and high sensitivity form is activated by a low calcium concentration (2-75 μM), whereas the latter and the lower sensitivity form is activated by higher concentrations of calcium (50-800 μM). These two isoforms have been studied extensively because they contribute most to the overall Ca²⁺-dependent proteolysis inflicted during patho-physiology. Upon calcium binding and activation, the two main isoforms and potentially other similar calpains become the targets of their endogenous inhibitor, calpastatin, which potently and specifically inhibits their activity. Calpastatin contains four inhibitory repeats, each of which independently binds a calpain molecule in its active, Ca²⁺-bound conformation with high affinity.

Calpains play important roles in various physiological processes. Under normal physiological conditions, calpains specifically and effectively target a plethora of proteins central to a multitude of signaling pathways involved in cell cycle progression, cell death, cell migration, insulin secretion, muscle homeostasis, platelet activation, and NF-KB activation. Elevated calpain levels, or low calpastatin levels, are also implicated in the patho-physiology of heart and neuronal degeneration, muscular dystrophy, cataract progression, inflammation (e.g. rheumatoid arthritis), and cancer.

Proteolysis by the calpains has been established as a major contributor to muscle protein degradation (Goll, et al. (2008) J. An. Sci. 86:E19-35) in patho-physiology as well as a key regulator of postmortem partial muscle degradation associated with meat tenderization (essentially loosening up the muscle fibers, myofibrils; Kemp, et al. (2010) Meat Sci. 84:248-56). Combined, these roles have the overall potential to improve the quality of meats, by promoting an increase in muscle mass without the compromise in the most important trait for customers, meat tenderness.

The crystal structures of rat (Hosfield, et al. (1999) EMBO J. 18:6880-6889) and human (Strobl, et al. (2000) Proc. Natl. Acad. Sci. USA 97:588-92) m-calpain heterodimers determined in the absence of Ca²⁺ have revealed a circular arrangement of domains. The circle extends from the anchor peptide ˜20 residues) at the N terminus of the large subunit (80 kDa), through the cysteine protease region (domains I ˜190 residues and II ˜145 residues), along the C2-like domain III ˜160 residues), down the linker (˜15 residues) and into the EF-hand-containing domain IV (˜170 residues). Domain IV makes intimate contacts with the homologous 28 kDa small subunit (domain VI) through pairing of their fifth EF-hands, and the small subunit completes the ring by binding to the anchor peptide. Domain V of the small subunit is invisible in the human heterodimer structure likely due to intrinsic disorder brought about by its high content of glycine residues. In this circular structure, domains I and II are held slightly apart and miss-aligned such that the active site cleft is too wide for catalysis. Activation by Ca²⁺ must realign domains I and II to bring the catalytic residues in register for peptide bond hydrolysis. However, in the absence of a Ca²⁺-bound crystal structure the mechanism of activation of calpain has been controversial (Sorimachi & Suzuki (2001) J. Biochem. (Tokyo) 129:653-664).

Various disclosures have suggested the use of the calpain structure to identify inhibitors. For Example, U.S. Pat. No. 7,236,891 describes a method for designing a ligand that binds to one or more domains of a calpain by crystallizing domains I and II of calpain in the presence of a cation, analyzing structural features of the crystallized domains I and II, and using the structural information to design a ligand having the ability to bind to domains I and II in the presence of the cation. This reference teaches that ligands identified and/or designed according to the method disclosed therein can be used to treat diseases or disorders such as cardiovascular disorder, Alzheimer's disease and other disorders that involve cation-dependent polypeptides or enzymes.

In addition, approaches have been suggested for activating calpain. For example, U.S. Pat. No. 6,042,855 discloses the use of vitamin D to stimulate calcium-activated calpain activity and improve the tenderness of meat and meat products. Similarly, U.S. Patent Application No. 2005/0053693 describes the use of a source of dietary anions to improve serum levels of calcium ions and increase intracellular levels of calcium, which in turn leads to accelerated calpain activity.

Mutations in the calpain and calpastatin loci have also been associated with meat tenderness. For example, a specific single nucleotide polymorphism (SNP) in the gene encoding μ-calpain has been shown to affect meat tenderness in bovine (see U.S. Pat. No. 7,238,479). Similarly, a single nucleotide polymorphism within intron 5 of the bovine CAST locus encoding the calpastatin protein has been shown to be associated with post-mortem muscle tenderness (see U.S. Patent Application No. 2006/0211006. In addition, U.S. Patent Application No. 2007/0172848 discloses a variety of markers associated with the quality of porcine meat. These markers include a SNP representing a shift from an arginine codon (AAA, Allele 2) to lysine (AGA, Allele 1) in exon 13 domain 1 of the CAST gene; a SNP representing a change from an arginine codon (AGA) to a serine codon (AGC Allele 1) in exon 28 (domain 4) of the CAST gene; a SNP representing a change from a threonine codon (ACT, Allele 1) to an alanine codon (GCT) in exon 22 (domain 3) of the CAST gene; and a SNP resulting in a change from a asparagine codon (AAT, Allele 1) to a serine codon (AGT) in exon 6 (domain L) of the CAST gene.

### SUMMARY OF THE INVENTION

The present invention features stabilized and destabilized variant calpastatin proteins and stabilized variant calpain proteins and fusions of the same for use in methods of activating/inactivating Or stabilizing/destabilizing calpain, enhancing muscle growth and facilitating the tenderization of meat. In some embodiments, a variant calpastatin contains an insertion or deletion in the occluding loop of inhibitor region B, whereas in other embodiments, a variant calpastatin is truncated. In still other embodiments, a variant calpastatin includes domains A, B and C of the inhibitory repeat 1, repeat 2, repeat 3, or repeat 4, wherein particular embodiments feature the modification of the sequences between domain A, B and/or C to enhance protease resistance or sensitivity. Likewise, a modified calpain with enhanced protease resistance is contemplated. In certain embodiments, the sequence of the occluding loop of inhibitor region B of the variant calpastatin is listed in Table 1. Isolated nucleic acid molecules, vectors, host cells, and transgenic non-human animals that express a variant calpastatin or variant calpain of the invention are also provided.

### BRIEF DESCRIPTION OF THE DRAWINGS

FIG. 1 is a schematic diagram of rat m-calpain and calpastatin. Recombinant calpain, composed on an intact 80 kDa catalytic subunit and a truncated 21 kDa regulatory subunit, binds 10 Ca²⁺ ions (spheres) to become activated. It forms stable Ca²⁺-dependent complexes with repeat 1 of calpastatin. The catalytic mutation of Cys105 to Ser did not influence the active site geometry in any of the previously determined calpain structures. DV of calpain and the L domain and repeats 2-4 of calpastatin were absent in the crystallized complex.

FIG. 2 shows calpastatin binding at the peripheral DIV and DVI. FIG. 2A, Detailed view of region A binding at DIV. FIG. 2B, Detailed view of region C binding at DVI. Hydrogen bonds are represented by dashed lines.

FIG. 3 shows a detailed view of the interaction between calpastatin and the catalytic cleft in the protease core DI-II. At the subsite P1, calpastatin distorts from the substrate path and projects residues 174-178, which kink between the P2 and P19 anchor sites.

FIG. 4 shows a detailed view of the interaction between calpastatin and a surface-accessible groove in DIII. Hydrogen bonds are represented as dashed lines.

FIG. 5 shows the primary sequence of calpastatin (SEQ ID NO:1), wherein Lys¹²⁵ and Lys²³⁶ delimit the trypsin-resistant fragment of calpastatin in the complex with m-calpain. Sequence identity between the four inhibitory repeats of rat, human, mouse, pig and chicken calpastatin is boxed (100%), double underlined (≧75%) and single underlined (50%). The 99-residue (128-226) calpastatin is delimited by filled arrowheads. The AB and BC constructs end and start at the diamond and circle arrow, respectively, sharing termini (open arrowheads) with the 86-residue (134-219) full-length construct that crystallized in complex with calpain.

FIG. 6 shows a detailed view of the interaction between the basic region in DIII and the active site.

FIG. 7 depicts the calpain-calpastatin proteolytic system. A schematic diagram illustrating the Ca²⁺-induced activation of calpain and its inhibition by calpastatin. DIII has a fundamental role in relaying the Ca²⁺-induced structural changes (dotted arrows) from the peripheral domains to the catalytically competent yet labile protease core. Concerted binding of the intrinsically unstructured protein (IUP) calpastatin to peripheral domains and the active site of calpain results in low-nanomolar inhibition.

### DETAILED DESCRIPTION OF THE INVENTION

The 3.0 Å crystal structure of Ca²⁺-bound m-calpain in complex with the first calpastatin repeat, both from rat, has now been determined revealing the mechanism of exclusive specificity. The structure highlights the complexity of calpain activation by Ca²⁺, illustrating key residues in a peripheral domain that serve to stabilize the protease core on Ca²⁺ binding. Fully activated calpain binds ten Ca²⁺ atoms, resulting in several conformational changes allowing recognition by calpastatin. The crystal structure of the calpain-calpastatin complex revealed how calpastatin uses three regions to interact with calpain: the N- and C-terminal regions bind peripheral calpain domains, and the central region extensively occludes the active site groove to prevent access of substrates. The active site occlusion depends on a critical calpastatin loop, which avoids proteolysis by skipping over the active site cysteine, being stabilized in this conformation by extensive binding of the calpastatin flanking regions to the calpain protease core. The length of the loop is conserved, and mutagenesis to increase or decrease its size results in conversion of the inhibitor to a substrate. Moreover, by binding to each of the five globular domains of calpain, calpastatin wraps around an otherwise extremely vulnerable enzyme, protecting it from inactivating autolysis and even degradation by other proteases. Taken together, the ability of calpastatin to protect calpain and the engineered conversion from an inhibitor to a substrate make structure-based variant calpastatins ideal proteinaceous candidates for use as calpain activators/stabilizers. Accordingly, the present invention embraces variant calpastatins for use in activating and/or stabilizing calpain. Engineering of the calpain-calpastatin system in animal models, by converting calpastatin from an inhibitor to a stabilizer/activator will facilitate the identification of this system in patho-physiology and improve meat tenderization in livestock. Moreover, having identified regions of stability, calpastatin can be modified to produce stabilized/destabilized or activated/inactivated variants of use in modulating muscle growth. Likewise, variant calpain enzymes can be generated to further stabilize/destabilize the calpain-calpastatin complex.

Variant calpastatins of the invention (i.e., calpain activators/inactivators, calpain stabilizers/destabilizers, or stabilized/destabilized calpastatin), include recombinant calpastatin proteins or peptides containing substitutions, insertions or deletions in the occluding loop of inhibitor region B and within the intrinsically unstructured regions connecting regions A, B and C. Variant calpastatin proteins include full-length calpastatin, i.e., a calpastatin containing domain L and four repetitive calpain-inhibition domains (repeat domains 1-4), or truncated versions of calpastatin containing at least the occluding loop of inhibitor region B. Examples of full-length wild-type calpastatin protein sequences are provided in GENBANK Accession Nos. NP_{—}001741 (791 amino acid residue human isoform a), NP_{—}033947 (754 amino acid residue mouse protein), NP_{—}445747 (713 amino acid residue rat protein), NP_{—}001025489 (799 amino acid residue bovine protein), XP_{—}424713 (768 amino acid residue chicken protein), NP_{—}999232 (781 amino acid residue porcine protein), NP_{—}001075739 (718 amino acid residue rabbit protein) and NP_{—}001009788 (723 amino acid residue ovine protein). For the purposes of the present invention, a full-length calpastatin is intended to mean that, with the exception of the occluding loop of domain B, which can contain insertions or deletions, the remainder of the calpastatin protein is the same length as wild-type calpastatin protein.

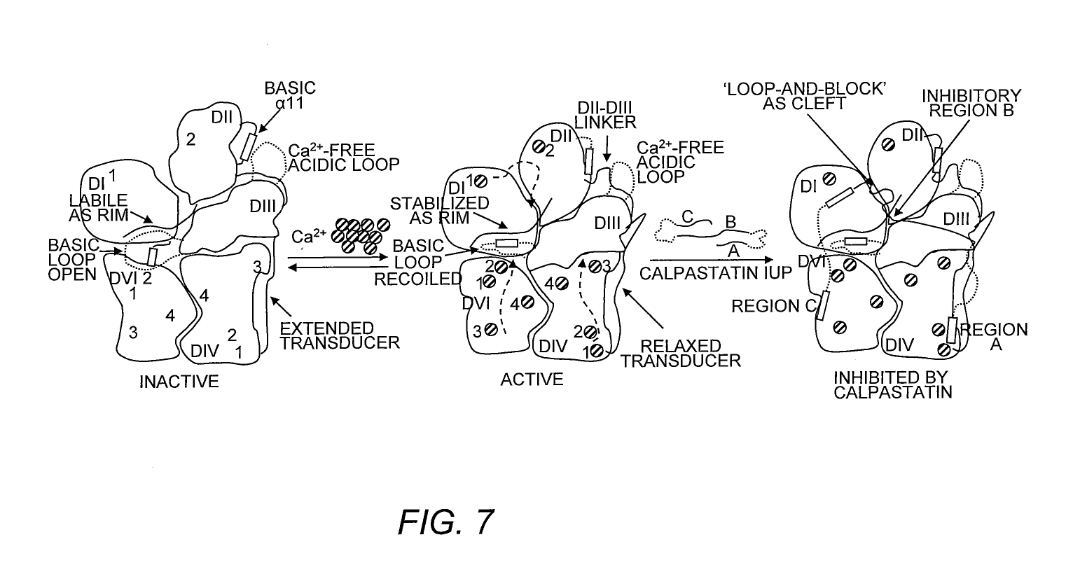

In some embodiments, truncated variant calpastatins are embraced by this invention. In one embodiment, a truncated variant calpastatin encompasses peptides (e.g., 5 to 100 amino acid residue fragments of calpastatin) that contain the occluding loop of inhibitor region B. In other embodiments, a truncated variant calpastatin encompasses versions of calpastatin that contain the occluding loop of inhibitor region B. In other embodiments, a truncated variant of calpastatin includes the occluding loop of inhibitor region B and sequences from one or more of domain A, domain B, domain C. In certain embodiments of this invention, domains A, B and/or C can be obtained from inhibitory repeat 1, repeat 2, repeat 3, or repeat 4. In particular embodiments, a truncated variant of calpastatin encompasses repeat 1. In so far as calpastatin binds to each of the five globular domains of calpain thereby protecting it from inactivating autolysis and even degradation by other proteases (FIG. 7), particular embodiments embrace stabilization of the calpain protein with a truncated version of calpastatin consisting of repeat 1 or domains A, B and C.

As will be understood by one skilled in the art upon reading the instant disclosure, the sequences between domains A, B, and C (i.e., interdomain regions) can be that of wild-type calpastatin or can be similar or different sequences of similar or different lengths so that binding to and stabilization of calpain is maintained or further improved. In this respect, particular embodiments of the present invention embrace modification of the sequences between domain A, B, and/or C to improve or enhance protease resistance of the variant calpastatin. By way of illustration, the sequences between domain A, B, and/or C of wild-type calpastatin can be analyzed for the presence of known protease recognition sequences and subsequently mutated to remove said recognition sequences. Such mutations include conserved amino acid substitutions to eliminate protease recognition or deletion of one or more amino acid residues of the recognition sequence. Protease recognition sequences are well-known in the art and available from the MEROPS peptidase database (Rawlings, et al. (2008) Nucleic Acids Res 36:D320-D325). Alternatively, the interdomain regions can be mutated based upon the results described herein so that binding and stabilization of calpain is decreased, reduced or eliminated. In this respect, known protease recognition sequences can be introduced into the interdomain regions to increase protease cleavage of calpastatin thereby leaving calpain vulnerable to inactivating autolysis and/or degradation by other proteases

As indicated, variant calpastatin molecules of the invention include substitutions, insertions or deletions in the occluding loop of inhibitor region B. Residues of the occluding loop of inhibitor region B from a variety of animals are presented in Table 1.

TABLE 1

| Organism | Sequence of Occluding Loop | SEQ ID NO: |
|---|---|---|
| Rat | Gly-Ile-Lys-Glu-Gly | 2 |
| Human | Gly-Lys-Arg-Glu-Val | 3 |
| Mouse | Gly-Ile-Lys-Glu-Gly | 2 |
| Pig | Gly-Glu-Lys-Glu-Glu | 4 |
| Bovine | Gly-Lys-Arg-Glu-Ser | 5 |
| Ovine | Gly-Glu-Arg-Asp-Asp | 6 |
| Chicken | Gly-Lys-Arg-Glu-Gly | 7 |
| Rabbit | Gly-Glu-Arg-Asp-Asp | 6 |

Substitutions, insertions or deletions of the occluding loop of calpastatin result in the activation/inactivation and/or stabilization/destabilization of calpain. Amino acid substitutions include those that alter amino acid side chains or structure of the occluding loop. By way of illustration, alanine substitutions would alter the charge and side chain length of, e.g., lysine or arginine residues, whereas substitutions with one or more proline residues would alter the structure of the loop. In particular embodiments, variant calpastatin molecules have an insertion or deletion in the occluding loop. For the purposes of the present invention, a deletion of the occluding loop encompasses removal of one, two, three, four or all five residues of the occluding loop, wherein consecutive or non-consecutive residues can be removed. An amino acid residue insertion is intended to mean the insertion of one or more, e.g., 10, 12, 14, 16, 18, or up to 20 amino acid residues in the occluding loop. Amino acid residues of the insertion can be random peptide sequences or can be a peptide, which upon cleavage (e.g., by an endogenous protease), provides a health or therapeutic benefit.

In some embodiments, the variant calpastatin of the invention is produced as a fusion protein. In one embodiment, the fusion protein is composed of a variant calpastatin fused to calpain to produce a single polypeptide that can fold more efficiently than the individual components, thereby resulting in a stabilized variant calpastatin and stabilized/activated calpain. Calpastatin fusion proteins containing calpain include calpain-calpastatin and calpastatin-calpain fusion proteins, wherein the orientation is indicative of N- and C-termini.

In other embodiments, the variant calpastatin or variant calpastatin fusion protein can penetrate cells and activate/stabilize or inactivate/destabilize calpain. Such proteins can include a cell-penetrating sequence (e.g., the signal sequence of Kaposi's fibroblast growth factor (kFGF)) and a variant calpastatin. In other embodiments, the variant calpastatin or variant calpastatin fusion protein contains sequences that facilitate the isolation and/or purification of variant calpastatin. Such fusions can include, e.g., glutathione S-transferase or an affinity tag, and a variant calpastatin.

A variant calpastatin or variant calpastatin fusion protein may be produced by cultured cells (e.g., E. coli, yeast, insect cells, or animal cells) transfected with nucleic acid molecules that encode the variant calpastatin or variant calpastatin fusion protein and have appropriate expression control sequences (see, e.g., U.S. Pat. No. 5,648,244). The nucleic acid molecules can be introduced into the cultured cells by standard transfection techniques, and the recombinantly produced variant calpastatin or variant calpastatin fusion protein can then be extracted and purified by techniques well-known in the art (e.g., immunoaffinity purification). It is well within the ability of one of ordinary skill in the art to carry out cloning and expression of a recombinant protein.

A variant calpastatin or variant calpastatin fusion protein can also be produced in significant amounts (i.e., in amounts sufficient for commercial or experimental use) by chemical synthesis. For example, a variant calpastatin or variant calpastatin fusion protein can be synthesized using solid phase N-(9-fluorenyl) methoxycarbonyl/N-methylpyrrolidone (Fmoc) chemistry (Jacobs, et al. (1994) J. Biol. Chem. 269:25494-25501). Purity can be assessed by HPLC and the correct molecular mass and protein sequence can be determined by mass spectrometry and Edman degradation. Peptide concentrations can be determined by quantitative amino acid analysis.

The ability of variant calpastatins (including fusion proteins) to activate/inactivate or stabilize/destabilize calpain can be assessed as described herein or in other assays known in the art (see, e.g., Bronk, et al. (1993) Am. J. Physiol. 264:G744-751, or modified versions thereof). For instance, calpain activity can be monitored in intact cells by measuring Ca²⁺ ionophore-specific peptidyl hydrolysis of the peptidyl-7-amino bond of a calpain substrate (e.g., Suc-LLVY-AMC or Suc-LLVY-aminoluciferin). To assay calpain activity in this way, cells are washed and re-suspended in HEPES-buffered (10 mM HEPES-NaOH, pH 7.4) Hank's balanced salts solution (without Ca²⁺ at about 2.5×10⁵ cells/ml and placed on ice. To assay calpain activity, the cell suspension is pre-warmed to 37° C. with stirring in an SLM ALMINCO 8000 fluorimeter. At t=−1 minute, ionomycin in DMSO (at a final concentration of 2.5 μM) or DMSO alone (negative control) is added to the cells. At t=0 minute, substrate is added to a final concentration of 50 μM. The initial rate of substrate cleavage, which is linear, is measured by spectroscopy at 2 to 3 minutes. The excitation wavelength is 360 ±2 nm and the emission detection wavelength is 460 ±10 nM. The ionomycin-dependent rate of substrate cleavage is subtracted from the ionomycin-independent rate of substrate cleavage to obtain the Ca²⁺-dependent rate.

A variant calpastatin or variant calpastatin fusion protein of the invention finds application in activating/inactivating or stabilizing/destabilizing calpastatin, e.g., in the study of the pathophysiology of diseases where calpain plays a role. Such diseases include, but are not limited to coronary thrombosis in coronary bypass surgery, vascular thrombosis and restenosis in angioplasty, the progression of an infarct in the event of myocardial infarction or stroke, subarachnoid hemorrhage or vasospasm, muscular dystrophy, cataracts, sickle cell crisis, HIV infection, Alzheimer's Disease, brain aging, traumatic brain injury, joint inflammation, arthritis and cancer. Moreover, variant calpastatin or variant calpastatin fusion protein of the invention can be used in the analysis of muscle growth and development.

A variant calpastatin or variant calpastatin fusion protein of the invention also finds application in meat tenderization, either in vitro or in vivo. Meat tenderization is largely dependent on the ratio between calpain and calpastatin. Postmortem in meat, as the cellular energy levels decrease, the intracellular calcium levels increase and intracellular stores begin to leak, flooding the cytoplasm. Calpains become activated but are prevented from ensuing random proteolysis by calpastatin, which typically is expressed in excess over the enzyme. Therefore, structure-based engineered calpastatins as calpain stabilizers/activators can be used to stabilize the proteolytic calpain-calpastatin complex to tenderize meat postmortem.

A variant calpastatin or calpastatin fusion may be used to enhance or to compete with the endogenous inhibitor. One of the problems facing the meat industry over the next half a century is the increased demand of meat production as the world's population will reach over nine billion people. Given that calpain proteolysis in the muscle has been established to reduce muscle mass (Costelli, et al. (2005) Int. J. Bchm. Cell Biol. 37:2134-46), strategies to block this proteolytic system during muscle growth will result in enhanced muscle growth. To overcome the opposing needs for inhibition of the calpain proteolytic system during muscle growth and for its activation/stabilization during postmortem meat tenderization, use of a calpastatin-calpain fusion protein for example ensures that postmortem a calpain protease, provided by the fusion, is available for meat tenderization. During normal muscle physiology, when Ca²⁺ signaling is tightly regulated, the activity provided by a calpastatin-calpain fusion will likely not contribute to extensive proteolysis as observed postmortem when intracellular Ca²⁺ levels are dysregulated.

For in vitro applications, the variant calpastatin or variant calpastatin fusion protein can be isolated and/or purified (e.g., to 80, 85, 90, 95, or 99% homogeneity) and be applied to a meat product (or livestock animal post-mortem) to facilitate activation and/or stabilization of calpain thereby enhancing or facilitating meat tenderization. In this respect, for certain application, the variant calpastatin or variant calpastatin fusion protein can be produced and isolated using conventional eukaryotic or bacterial expression systems. Thus, the invention encompasses nucleic acid molecules that encode a variant calpastatin or variant calpastatin fusion protein. The nucleic acid molecules can be inserted into vectors, such as those described herein, which will facilitate expression of the gene in a host cell. Accordingly, expression vectors containing such nucleic acid molecules and host cells transfected with these vectors are within the scope of the invention. A transformed cell is any cell into which (or into an ancestor of which) a nucleic acid molecule encoding a polypeptide of the invention has been introduced (e.g., by recombinant DNA techniques).

An isolated molecule of the invention is a molecule that has been removed from its natural environment. For example, a nucleic acid molecule is a nucleic acid molecule that is separated from its naturally occurring genome. Isolated nucleic acid molecules include nucleic acid molecules which are not naturally occurring, e.g., nucleic acid molecules created by recombinant DNA techniques. Nucleic acid molecules include both RNA and DNA, including cDNA and synthetic DNA (i.e., chemically synthesized DNA).

Expression systems that can be used to produce variant calpastatin or variant calpastatin fusion protein include, but are not limited to, microorganisms such as bacteria (for example, E. coli or B. subtilis) transformed with recombinant bacteriophage DNA, plasmid DNA, or cosmid DNA expression vectors containing the nucleic acid molecules of the invention; yeast (for example, Saccharomyces or Pichia) transformed with recombinant yeast expression vectors containing the nucleic acid molecules of the invention; insect cell systems infected with recombinant virus expression vectors (for example, baculovirus); or animal cell systems (for example, COS, CHO, BHK, 293, VERO, HeLa, MDCK, WI38, and NIH 3T3 cells) harboring recombinant expression constructs containing promoters derived from the genome of animal cells (for example, the metallothionein promoter) or from animal viruses (for example, the adenovirus late promoter and the vaccinia virus 7.5K promoter).

In bacterial systems, any conventional expression vector can be selected depending upon the use intended for the gene product being expressed. Such vectors include, but are not limited to, the E. coli expression vector pUR278 (Ruther et al. (1983) EMBO J. 2:1791), in which the coding sequence of the insert can be ligated individually into the vector in frame with the lacZ coding region so that a fusion protein is produced; pIN vectors (Inouye & Inouye (1985) Nucleic Acids Res. 13:3101-3109; Van Heeke & Schuster (1989) J. Biol. Chem. 264:5503-5509); and the like. pGEX vectors can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhidrosis virus (AcNPV) can be used as a vector to express foreign genes. The virus grows in Spodoptera frugiperda cells (for example, see Smith et al. (1983) J. Virol. 46:584; U.S. Pat. No. 4,215,051).

In animal host cells, a number of viral-based expression systems can be utilized. In cases where an adenovirus is used as an expression vector, the nucleic acid molecule of the invention can be ligated to an adenovirus transcription/translation control complex, for example, the late promoter and tripartite leader sequence. This chimeric gene can then be inserted in the adenovirus genome by in vitro or in vivo recombination.

Specific initiation signals may also be required for efficient translation of inserted nucleic acid molecules. These signals include the ATG initiation codon and adjacent sequences. The initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al. (1987) Methods in Enzymol. 153:516-544). Expression constructs capable of expressing variant calpastatin or variant calpastatin fusion proteins can be prepared using methods routinely practiced in the art. See, e.g., Sambrook & Russell (2001) Molecular Cloning: A Laboratory Manual, 3^{rd} Edition, Cold Spring Harbor Laboratory Press and other conventional laboratory manuals.

For in vivo applications, the variant calpastatin or variant calpastatin fusion protein can be administered to an animal any number of days or weeks prior to slaughter, e.g., by intramuscular injection. Alternatively, the calpain activator/stabilizer can be transgenically expressed by the animal. In this respect, the present invention also embraces a non-human transgenic animal that expresses a variant calpastatin or variant calpastatin fusion protein of the invention. For the purposes of the present invention, expression can be transient or stable. When expression is transient, the animal can be provided with a nucleic acid construct as described herein, any number of days or weeks prior to slaughter so that the variant calpastatin or variant calpastatin fusion protein is expressed in vivo. Wherein the variant calpastatin or variant calpastatin fusion protein construct is stably integrated into the non-human transgenic animal, expression can be controlled by an exogenous factor so as to limit meat tenderization to a number of days or weeks prior to slaughter. For example, the tetracycline-inducible promoter is conventionally used to regulate the expression of proteins via an exogenous factor (i.e., tetracycline).Non-human transgenic animals of the invention can be produced by any conventional method using, e.g., an expression construct described herein for expression in animal cells. For example, introduction of a nucleic acid molecule encoding a variant calpastatin or variant calpastatin fusion protein into the developing zygote or embryo (Brinster, et al. (1985) Proc. Natl. Acad. Sci. USA 82:4438-4442; U.S. Pat. No. 4,873,191) can be used to produce transgenic animals. Transgenic technology has been applied to both laboratory and domestic species for the study of human diseases (see, e.g., Synder, et al. (1995) Mol. Reprod. and Develop. 40:419-428), production of pharmaceuticals in milk (see, Ebert & Selgrath (1991) Changes in Domestic Livestock through Genetic Engineering, in Applications in Mammalian Development, Cold Spring Harbor Laboratory Press), develop improved agricultural stock (see, e.g., Ebert, et al., (1990) Animal Biotechnology 1:145-159) and xenotransplantation (see, e.g., Osman, et al. (1997) Proc. natl. Acad. Sci USA 94:14677-14682). In addition, microinjection of DNA into the nucleus can be used to generate transgenic offspring. Furthermore, a nucleic acid molecule encoding a variant calpastatin or variant calpastatin fusion protein can be directly delivered to a spermatogonium by infusing the nucleic acid molecule in situ into a testicle of a non-human animal (see U.S. Pat. No. 6,686,199). Introduction of nucleic acids encoding a variant calpastatin or variant calpastatin fusion protein can be via non-homologous or homologous recombination. Conventional approaches for homologous recombination and gene targeting in livestock are discussed in Laible & Alonso-Gonzalez (2009) Biotechnology J. 4:1278-1292. Non-human transgenic animals encompassed by the present invention include, but are not limited to, horses, cattle, pigs, goats, deer, rabbit, sheep, and poultry.

Non-human animals where the calpastatin gene has been replaced by a calpastatin variant including a calpastatin fusion protein using homologous recombination technologies (Laible and Alonso-Gonzalez (2009) Biotech. J. 4:1278-92) may be produced based on the technologies illustrated herein. In addition, one or more of the calpain genes that are known not to be stabilized by calpastatin may be replaced by homologous recombination to facilitate muscle growth and postmortem meat tenderization.

In so far as the in vitro or in vivo approaches for providing a stabilized variant calpastatin or stabilized variant calpastatin fusion protein to meat will improve meat tenderization, the present invention also embraces a method for facilitating the tenderization of meat by contacting a meat product (including an animal) with an effective amount of a variant calpastatin or variant calpastatin fusion protein so that tenderization of the meat product is facilitated. As discussed herein, contact of a meat product (including an animal) can be via intramuscular injection or transgenic expression, wherein an effective amount of a variant calpastatin or variant calpastatin fusion protein is an amount which measurably activates or stabilizes calpain to substantially decrease shear force of meat in comparison to untreated meat. Indeed, it is expected that treatment in accordance with the present invention will improve the tenderness of the tougher steaks and roasts from the round and chuck.

As an alternative to activating/stabilizing calpain, the present invention also features methods for facilitating muscle growth by inactivating/destabilizing calpain. In accordance with this embodiment, muscle tissue is contacted with a destabilized variant calpastatin or destabilized variant calpastatin fusion protein with, e.g., one or more protease recognition sequences in one or more loops between the domains A, B and C, so that muscle growth is measurably increased or enhanced. In this respect, muscle degradation is reduced or decreased in the muscle tissue contacted with the destabilized variant calpastatin or destabilized variant calpastatin fusion protein as compared to muscle tissue which has not be contacted with the destabilized variant calpastatin or destabilized variant calpastatin fusion protein. The invention embraces both in vivo and in vitro aspects of facilitating muscle growth with intramuscular or topical delivery and transgenic expression of the destabilized variant calpastatin or destabilized variant calpastatin fusion protein included within the scope of the invention.

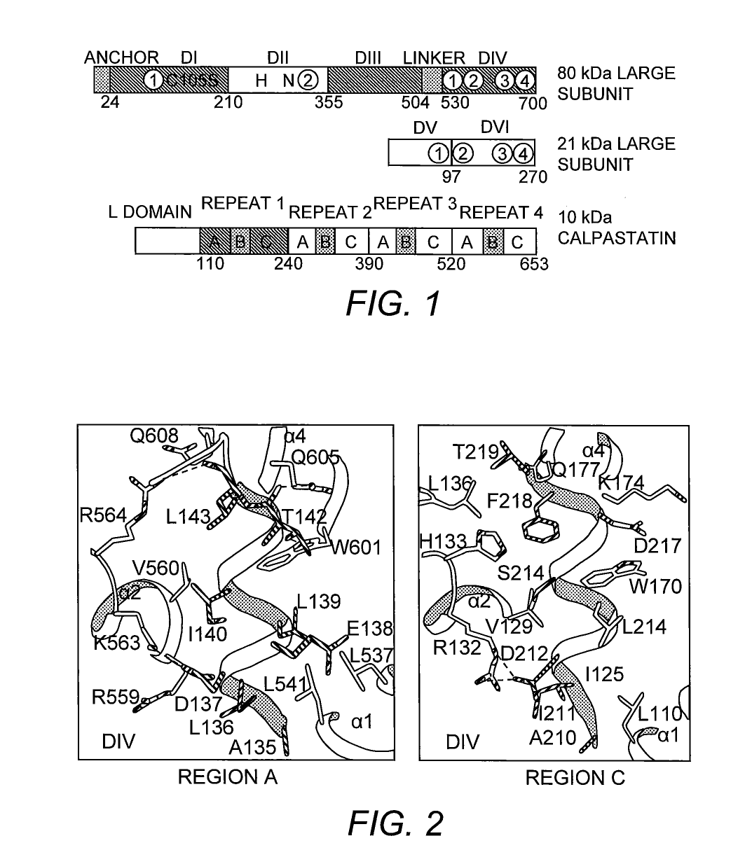

As a further embodiment of this invention, the calpain protein can also be mutated/modified to produce a variant calpain, which stabilizes or further stabilizes the calpain-calpastatin complex or calpain-calpastatin fusion protein. By way of illustration, amino acid residues of calpain that interact with calpastatin (see FIGS. 1-4 and 6) can be analyzed by computer modeling, random mutation, and/or selective point mutation using routine methods to produce variant calpain proteins that are more stable than wild-type calpain when in complex with a wild-type calpastatin or variant calpastatin protein of the invention. Moreover, the primary sequence of calpain can be analyzed for protease recognition sequences, wherein such sequences can be removed to enhance protease resistance. In one embodiment, the variant calpain is a variant of m-calpain. In another embodiment, the variant calpain is a variant of μ-calpain. In a further embodiment, the variant calpain is a variant of calpain-3 to calpain-15. Protein sequences of wild-type calpains are well-known in the art. For example, the protein sequences of the small subunit, μ-calpain and m-calpain are available under the GENBANK Accession Nos. listed in Table 2.

TABLE 2

| Calpain | Organism | Accession No. |
|---|---|---|
| Small Subunit | Human | NP_001740 |
| | Mouse | NP_033925 |
| | Rat | NP_058814 |
| | Bovine | NP_776686 |
| | Ovine | |
| | Chicken | XP_001232969 |
| | Porcine | NP_001087910 |
| | Rabbit | NP_001075733 |
| Catalytic-1 | Human | NP_005177 |
| (μ/I) Subunit | Mouse | NP_001103974 |
| | Rat | NP_062025 |
| | Bovine | NP_0776684 |
| | Ovine | NP_001120739 |
| | Chicken | NP_990634 |
| | Porcine | NP_999137 |
| Catalytic-2 | Human | NP_001739 |
| (m/II) Subunit | Mouse | NP_033924 |
| | Rat | NP_058812 |
| | Bovine | XP_869198 |
| | Ovine | NP_001106288 |
| | Chicken | NP_990411 |
| | Porcine | NP_001093658 |

Stabilization and activity of the calpain-calpastatin complex can be assessed using the methods described herein or any conventional method. As described for variant calpastatin, fusion proteins, expression vectors, host cells, and transgenic non-human animals can be produced with the variant calpain of this invention.

The invention is described in greater detail by the following non-limiting examples.

### Example 1: Materials and Methods

Cloning, Mutagenesis, Peptide Synthesis, Protein Expression and Purification. The rat calpastatin repeat 1 clone encoding residues Met¹¹⁹-Ser²³⁸ (gi 13540322) was cloned as an N-terminally His₁₀-tagged construct in pET16b vector. Subsequent cloning in the NcoI and XhoI sites of the kanamycin-resistant pET24d vector was performed using this vector as PCR template and the following oligonucleotides as primers, with a stop codon engineered to exclude the C-terminal His₆ tag: 5′med 5′-GCA TGG CCA TGG ACA AGT CAG GCG TGA ATG CTG-3′ (SEQ ID NO:8), 3′med 5′-GTG GTG CTC GAG TTA CTT TCC AGT TGG AGA GCT ACA G-3′ (SEQ ID NO:9), 5′sh 5′-GCA TGG CCA TGG CTG CTT TGG ATG ACC TGA TAG-3′ (SEQ ID NO:10), 3′sh 5′-GTG GTG CTC GAG TTA GGT GAA ATC AGA TGA CCA GGC A-3′ (SEQ ID NO:11), 5′BC 5′-GCA TGG CCA TGG ACC CAA TGG ATT CTA CCT AC-3′ (SEQ ID NO:12) and 3′BC 5′-GTG GTG CTC GAG TTA ACA GGT GAA ATC AGA TGA CAA GGC-3′ (SEQ ID NO:13). Medium-sized (Met-Asp¹²⁸-Lys²²⁶) and short (Met-Ala¹³⁴-Thr²¹⁹) calpastatin repeat 1 constructs were produced and as was peptide BC (Met-Asp¹⁶³-Cys²²⁰) In the short construct, a stop codon was introduced after Lys¹⁹⁰ using the QUICK CHANGE protocol (Stratagene) and the forward primer 5′-GAA ACT TCT GGA GAA ATA AGA AGC TAT CAC AGG-3′ (SEQ ID NO:14) (reverse not shown) to generate peptide AB (Met-Asp¹²⁸ Lys¹⁹⁰). The E. coli BL21 DE3 strain was used to express all five derivatives of calpastatin. In all calpastatin constructs the initiating Met was removed during expression as indicated by intact mass determination by mass spectrometry. The m-calpain heterodimer, C105S m80 kDa/21 kDa, which lacks the glycine-rich DV, and the protease core from μ-calpain (μI-II) were expressed in E. coli and purified according to established methods (Moldoveanu, et al. (2002) Cell 108:649-660; Elce, et al. (1995) Protein Eng. 8:843-848). Forward mutagenesis primers for the 80 kDa subunit included R417A 5′-CCA GAA GCA TCG GGC GCG GCA GAG GAA-3′ (SEQ ID NO:15), R420A 5′-GGC GGC GGC AGG CGA AGA TGG GTG AG-3′ (SEQ ID NO:16) and R469A 5′-CCT TCA TCA ACC TCG CGG AGG TCC TCA AC-3′ (SEQ ID NO:17), and for calpastatin K176Δ 5′-GGC ACT GGG TAT AGA AGG GAC TAT TCC-3′ (SEQ ID NO:18), E177Δ 5′-GCA CTG GGT ATA AAA GGG ACT ATT CCT C-3′ (SEQ ID NO:19) and K176/E177Δ 5′-GGC ACT GGG TAT AGG GAC TAT TCC TC-3′ (SEQ ID NO:20). For ¹⁵N-labeling and ¹³C, ¹⁵N-labeling, the medium-sized and short calpastatins were expressed in M9 medium (Sambrook, et al. (1989) Molecular Cloning: A Laboratory Manual, Cold Spring Harbor Laboratory Press) in the presence of ¹⁵N-NH₄C1 and ¹⁵N-NH₄C1 plus ¹³C-glucose, respectively. All forms of calpastatin were purified by boiling the cell lysate for 15 minutes, followed by Ni-NTA affinity chromatography (the original His-tagged construct), Q-SEPHAROSE and C18 reversed-phase HPLC or S200 gel filtration chromatography. All protein preparations were exchanged into 20 mM HEPES (pH 7.5), 5 mM DTT storage buffer, were flash frozen in liquid nitrogen, and stored at −80° C. Peptide B1 Ac-ALGIKEGTIPPEYRKLLE-NH₂ (SEQ ID NO:21) was synthesized and HPLC-purified.

Calpain-Calpastatin Complex Formation and Purification. The m-calpain-calpastatin complex was formed by slowly titrating 50 mM CaCl₂ into a solution (˜20-30 ml) containing purified calpain (10-20 mg) and a 2-5X molar excess of calpastatin in 50 mM HEPES buffer (pH 7.5) until the CaCl₂ concentration reached ˜5-10 mM. The subsequent steps of purification were performed in solutions containing 5 mM CaCl₂ to prevent dissociation of the complex. Excess untagged calpastatin was removed by recovering the complex on Ni-NTA (QIAGEN), which bound to the column using the C-terminal His₆-tag on the calpain large subunit. Further purification involved SEPHACRYL 5200 and Q-SEPHAROSE chromatography. The complexes were stored in 20 mM HEPES (pH 7.5), 5 mM DTT, 10 mM CaCl₂ at −80° C.

Limited Proteolysis and Autolysis of the Calpain-Calpastatin Complex. Limited proteolysis (Moldoveanu, et al. (2001) Biochim. Biophys. Acta 1545:245-254) or autolysis reactions were performed at 22° C. in a final volume of less than 150 μl, in 1 mM CaCl₂ and 50 mM HEPES at pH 7.5. Purified 1 mg/ml (9 μM) m-calpain-calpastatin complex was proteolysed by 0.001 mg/ml (0.04 μM) trypsin (Sigma). For autolysis, 1 mg/ml (10 μM) m-calpain was incubated at 1 mM CaCl₂ with wild-type or mutant full-length calpastatin or fragments AB and BC. At specific time intervals, aliquots were removed, the reaction was stopped by the addition of 2XSDS gel sample buffer, and the products were analyzed by SDS-PAGE.

Calpastatin Inhibition Assays. Hydrolysis assays by m-calpain (1 nM or 20 nM), m-calpain mutants (1 nM) and μI-II (1.25-2.5 μM) were performed in 100-μl volumes containing 50 mM HEPES (pH 7.5), 200 mM NaCl, 1 mM DTT, various concentrations of substrate (0-1.5mM SLY-MCA (Sigma) or 0-50 μg/ml BODIPY-casein (Invitrogen)) and increasing inhibitor concentrations: 0-50 nM wild-type or mutant full-length calpastatin, fragments AB and BC, 0-20 μM peptide B1 for m-calpain, and 0-750 μM peptide B1 for μI-II. Duplicates or triplicates were performed for each condition in 96-well plates using a Molecular Devices microplate reader.

NMR Assignments of Calpastatin Flexible Regions in Complex with Calpain. The complexes between ¹⁵N-labeled medium or short calpastatin and unlabeled calpain were subjected to ¹⁵N, ¹H HSQC analysis in the storage buffer supplemented with 10% D₂O at 25° C. The spectra were collected on a Bruker Avance DRX 600 MHz spectrometer equipped with a triple-resonance CryoProbe. HNCA and CBCA(CO)NH experiments with ¹⁵N/¹³C-labelled Asp¹²⁸-Lys²²⁶ calpastatin bound to unlabelled calpain were used to assign the mobile regions of the inhibitor. Spectral processing was done using NMRpipe (Delaglio, et al. (1995) J. Biomol. NMR 6:277-293) and spectral analysis using nmrView (Johnson (2004) Methods Mol. Biol. 278:313-352).

Calpain-Calpastatin Complex Crystallization and Structure Determination. After extensive screening and expansions, the complex between m-calpain and the NMR-trimmed Ala¹³⁴-Thr²¹⁹ calpastatin was crystallized in 4-9% PEG 3350, 5-10 mM CaCl₂ and 50-100 mM NaOAc (pH 5.5) using the hanging-drop method by mixing equal volumes of complex (10-15 mg/ml) and mother liquor. Cryo conditions included mother liquor and up to 30% ethylene glycol. The crystals were assigned to the tetragonal space group P4₂ with one molecule per asymmetric unit and diffracted to 2.8-3.5 Å at synchrotrons (Table 3). Multiple native data sets were collected at 5.0.1-3 beamlines of the Advanced Light Source, X29 beamline at Brookhaven National Laboratories, and at SERCAT 22BM and 22ID beamlines at the Advanced Photon Source. The structure was determined by molecular replacement using as search models the Ca²⁺-bound rat m-calpain protease core (1MDW), the DIII and DIV of Ca²⁺-free rat m-calpain (1DFO) and the Ca²⁺-bound small subunit of rat m-calpain (1DVI) and Phaser (McCoy, et al. (2007) J. Appl. Cryst. 40:658-674) included in the CCP4 package (Collaborative Computational Project, Number 4. (1994) Acta Crystallogr. D 50:760-763). Structural refinement was processed using Refmac5 (Winn, et al. (2001) Acta Crystallogr. D 57:122-133) and CNS (Brünger, et al. (1998) Acta Crystallogr. D 54:905-921), with manual fitting performed using Xfit (McRee (1992) J. Mol. Graph. 10:44-46).

TABLE 3

| | 2.95 Å (SERCAT 22ID) | | 3.1 Å (SERCAT 22BM) | |
|---|---|---|---|---|
| Data Collection | | | | |
| Space Group | P4₂ | | P4₂ | |
| Cell Dimensions | | | | |
| a, b, c (Å) | 147.3, 147.3, 47.2 | | 150.5, 150.5, 48.3 | |
| α, β, γ (°) | 90.0, 90.0, 90.0 | | 90.0, 90.0, 90.0 | |
| Resolution (Å) | 50.0-2.95 | (3.06-2.95)* | 50.0-3.1 | (3.2-3.1) |
| R_{sym} or R_{merge} | 9.6 | (82.0) | 7.1 | (51.3) |
| I/σI | 21.4 | (2.1) | 20.1 | (2.2) |
| Completeness (%) | 99.6 | (98.3) | 99.6 | (99.6) |
| Redundancy | 4.4 | (4.2) | 3.6 | (3.5) |
| Refinement | | | | |
| Resolution (Å) | 49.0-2.94 | | 50.0-3.1 | |
| No. Reflections | 20755 | | 18936 | |
| R_{work} or R_{free} | 22.9/29.9 | (5.1%) | 23.0/29.9 | (5.1%) |

| No. atoms | 7303 | | 7303 | |
|---|---|---|---|---|
| Protein | 7293 | | 7293 | |
| Ca²⁺ | 10 | | 10 | |
| B-factors | 94.7 | | 90.3 | |
| R.m.s. Deviations | | | | |
| Bond lengths (Å) | 0.012 | | 0.028 | |
| Bond angles (°) | 1.485 | | 1.816 | |
| Molprobity score^{#} | 3.28 (49^{th} percentile) | | | |
| | 3.2 Å (BNL X29) | | 3.5 Å (ALS 5.0.1) | |
| Data Collection | | | | |
| Space Group | P4₂ | | P4₂ | |
| Cell Dimensions | | | | |
| a, b, c (Å) | 148.3, 148.3, 47.7 | | 148.2, 148.2, 47.8 | |
| α, β, γ (°) | 90.0, 90.0, 90.0 | | 90.0, 90.0, 90.0 | |
| Resolution (Å) | 50.0-3.2 | (3.3-3.2) | 35.0-7.5 | (3.6-3.5) |
| R_{sym} or R_{merge} | 4.9 | (36.3) | 5.2 | (15.2) |
| I/oI | 16.6 | (1.6) | 26.0 | (6.2) |
| Completeness (%) | 94.0 | (85.7) | 93.8 | (79.2) |
| Redundancy | 2.4 | (2.3) | 4.0 | (3.1) |
| Refinement | | | | |
| Resolution (Å) | 50.0-3.2 | | 50.0-3.5 | |
| No. Reflections | 15716 | | 11897 | |
| R_{work} or R_{free} | 23.1/31.7 | (4.9%) | 21.1/27.6 | (5.0%) |

| No. atoms | 7303 | 7303 |
|---|---|---|
| Protein | 7293 | 7293 |
| Ca²⁺ | 10 | 10 |
| B-factors | 95.1 | 96.5 |
| R.m.s. Deviations | | |
| Bond lengths (Å) | 0.023 | 0.026 |
| Bond angles (°) | 1.830 | 1.973 |
| Molprobity score^{#} | | |

#Molprobity score was conventionally obtained (Davis, et al. (2007) Nucleic Acids Res. 35: W375-83).

### Example 2: Crystal Structure

The crystal structure of the complex between m-calpain and residues 134-219 of calpastatin inhibitory repeat 1 was determined (Table 3). The m-calpain heterodimer is composed of an 80 kilodalton (kDa) catalytic subunit and a 28 kDa regulatory subunit (Suzuki, et al. (2004) Diabetes 53:S12-S18). The large subunit contains the Ca²⁺-dependent protease core domain I-II (DI-II)(Moldoveanu, et al. (2002) supra), DIII (which resembles C2 domains involved in membrane targeting) and the Ca²⁺-binding penta-EF-hand DIV to heterodimerize the homologous DVI of the regulatory subunit (FIG. 1) (Hosfield, et al. (1999) EMBO J. 18:6880-6889). The catalytically inactive C105S 80 kDa subunit was used to overcome unwanted proteolysis observed with the wild-type protein (Elce, et al. (1995) supra). The regulatory subunit was substituted with the 21 kDa DVI (FIG. 1) (Elce, et al. (1995) supra). The 72 kDa calpastatin inhibits heterodimeric calpains with nanomolar affinity, being composed of a non-inhibitory 12 kDa leader L domain and four 15-kDa calpain inhibitory repeats (FIG. 1) (Wendt, et al. (2004) Biol. Chem. 385:465-472). Each repeat contains three regions (A-C) predicted to interact with calpain (Wendt, et al. (2004) supra). The calpain-calpastatin complex was optimized by truncating calpastatin from a longer construct (residues 119-238) on the basis of results from limited proteolysis and NMR spectroscopy, both of which identified the amino- and carboxy-terminal mobile regions that impeded crystallization (FIG. 1).

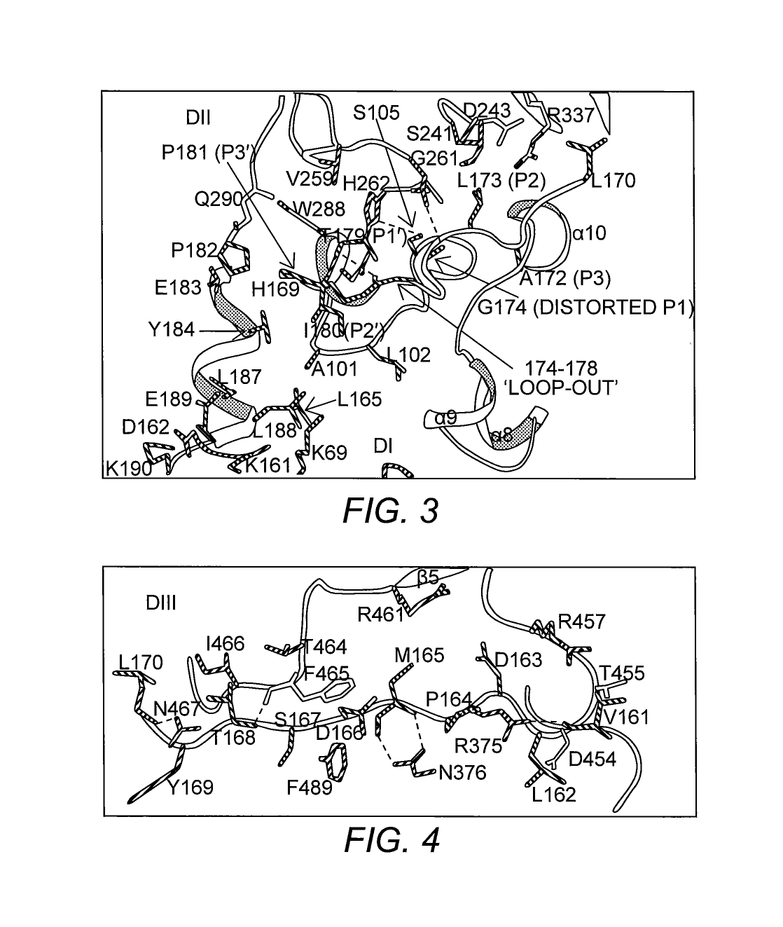

Calpastatin recognizes the Ca²⁺-induced conformation of m-calpain but does not coordinate Ca²⁺ in the complex. Regions A and C fold as amphipathic helices when bound to the Ca²⁺-induced hydrophobic pockets in the corresponding penta-EF-hand domains (FIG. 2). Previously, homology modeling based on the Ca²⁺-dependent complex between DVI and region C of calpastatin predicted the mode of binding for region A (Todd, et al. (2003) J. Mol. Biol. 328:131-146). Regions A and B of calpastatin engage sites in DIV and respectively. Region B associates with DI-III to obstruct the active site in the extended substrate-like orientation. Intrinsic disorder in the free state enables calpastatin to adapt structurally on binding to the substrate-binding cleft of calpain, distantly resembling the inhibitory conformation of the broad-specificity, structured protease inhibitors, the cystatins (Bode & Huber (2000) Biochim. Biophys. Acta 1477:241-252). Region B is anchored on either side of the active site C105S and avoids proteolysis by forcing a kink (Gly¹⁷⁴-Ile-Lys-Glu-Gly¹⁷⁸; SEQ ID NO:2) between the flanking residues, Leu¹⁷³ at the substrate binding S2 subsite and Thr¹⁷⁹ at S1′ (FIGS. 3 and 4). The unprimed and primed substrate binding subsites are N- and C-terminal, respectively, from the scissile bond P1-P1′. S and P distinguish protein and substrate subsites, respectively. N-terminal to the kink, residues Val¹⁶¹-Leu¹⁷⁰ participate in hydrogen bonds mediated by backbone atoms (Table 4) and hydrophobic interactions with the DIII surface that juxtaposes the active site (FIG. 4). The remainder of region B binds to the DI-II (FIG. 3). Ala¹⁷² marks the S3 site whereas the Leu¹⁷³ interaction is of particular importance at the S2 pocket, the main specificity determinant of calpains (Cuerrier, et al. (2005) J. Biol. Chem. 280:0632-40641; Cuerrier, et al. (2007) J. Biol. Chem. 282:9600-9611). The side chain of Leu¹⁷³ at S2 is superimposable to Leu² of leupeptin in complex with the protease core DI-II of μ-calpain, μI-II (Moldoveanu, et al. (2004) supra). C-terminal to the kink, region B engages the S19-S29-S39 subsites with residues Thr¹⁷⁹-Ile-Pro^{181.} The conformation of Pro¹⁸¹-Pro-Glu-Tyr¹⁸⁴ (SEQ ID NO:22) changes the direction as the Glu¹⁸³-Lys¹⁹⁰ helix targets DI.

TABLE 4
R1 Intramolecular Kink Stabilization

| | | |
|---|---|---|
| Glu¹⁷⁷ O | 3.2 | Ile¹⁷⁸ N |
| Lys¹⁷⁶ O | 3.3 | Thr¹⁷⁷ N |

R1 Interaction at DI-II

| R1 | Å | DI-II |
|---|---|---|
| Thr¹⁷⁹ OG1 | 2.4 | His²⁶² ND1 |
| Tyr¹⁸⁴ OH | 2.7 | His¹⁶⁹ ND1 |
| Gly¹⁷⁴ N | 3.0 | Gly²⁶¹ O |

R1 Interaction at DIII

| R1 | Å | DIII |
|---|---|---|
| Met¹⁶⁵ O | 2.6 | Asn³⁷⁶ ND2 |
| Val¹⁶¹ O | 2.6 | Arg³⁷⁵ NH1 |
| Met¹⁶⁵ N | 3.0 | Asn³⁷⁶ OD1 |
| Thr¹⁶⁸ N | 3.0 | Phe⁴⁶⁵ O |
| Leu¹⁷⁰ N | 3.2 | Asn⁴⁶⁷ OD1 |

R1 Interaction at DIV

| R1 | Å | DIV |
|---|---|---|
| Asp¹³⁹ OD1 | 2.4 | Lys⁵⁶³ NZ |
| Thr¹⁴⁴ OG1 | 2.8 | Trp⁶⁰¹ NE1 |
| Thr¹⁴⁴ O | 2.8 | Gln⁶⁰⁵ OE1 |
| Leu¹⁴⁵ O | 3.1 | Arg⁵⁶⁴ NH2 |

R1 Interaction at DVI

| R1 | Å | DVI |
|---|---|---|
| Asp²¹⁴ OD1 | 3.2 | Arg¹³² NH2 |
| Asp²¹⁴ OD1 | 3.2 | Arg¹³² NE |

DI-II-DIII Interface

| DIII | Å | DI-II |
|---|---|---|
| Glu⁴⁷⁰ OE1 | 2.4 | Glu²⁰⁵ OE2 |
| Arg⁴¹⁷ NH1 | 2.5 | Gly¹⁹⁷ O |
| Arg⁴¹⁷ NH2 | 2.7 | Glu²⁰² OE2 |
| Arg⁴²⁰ NH1 | 2.8 | Ser¹⁹⁶ O |
| Trp³⁵⁶ NE1 | 2.9 | Asp³⁴⁶ OD2 |
| Lys³⁶⁰ NZ | 3.0 | Gly¹⁴⁷ O |
| His⁴¹⁵ N | 3.0 | Tyr¹⁴⁶ OH |
| Arg⁵⁰⁰ NH2 | 3.1 | Gly²¹⁰ O |
| Arg⁴¹⁷ NH2 | 3.1 | Gly¹⁹⁷ O |
| Arg⁵⁰⁰ NH2 | 3.1 | Gly²⁰⁹ O |
| Arg⁴⁷⁴ NH2 | 3.2 | Glu²¹³ OE1 |
| Arg⁴⁶⁹ NH2 | 3.2 | Glu²⁰⁵ OE2 |
| Arg⁴¹⁷ NH1 | 3.2 | Ala¹⁹⁴ O |
| Arg⁴²⁰ NH2 | 3.2 | Ser¹⁹⁶ O |

DIII-DIV Interface

| DIII | Å | DIV |
|---|---|---|
| Ile⁴⁴⁶ O | 3.0 | Lys⁶²⁹ NZ |
| His⁴⁴⁷ ND1 | 3.0 | Tyr⁶²⁵ OH |

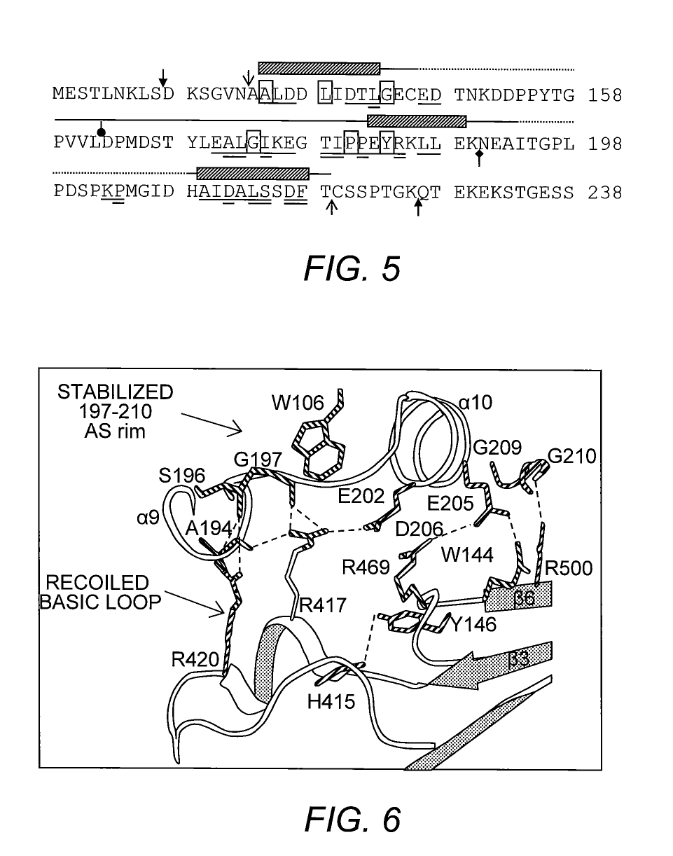

The mechanism of inhibition was determined by shortening the kink through deletion of Lys¹⁷⁶, Glu¹⁷⁷ or both. In all instances, the low nanomolar half-maximal inhibitory concentration (IC₅₀) values for m-calpain inhibition, derived from initial rate analysis, did not change significantly compared to wild type. However, all mutants succumbed to proteolysis within the kink, permitting catalytic cleft access and resulting in complete autoproteolysis of the complex within hours. The 5-residue kink is therefore essential to overcome proteolysis and its length has indeed been conserved (FIG. 5) (Wendt, et al. (2004) supra). An 18-residue peptide B1, specific for DI-II (Ala¹⁷²-Glu¹⁸⁹), inhibited m-calpain with an IC₅₀ of 410±80 nM (mean±s.e.m). It has been shown that a 27-residue peptide, corresponding to Asp¹⁶³-Glu¹⁸⁹ of region B, inhibited μ-calpain with an IC₅₀ of -30 nM (Betts, et al. (2003) J. Biol. Chem. 278:7800-7809). Residues Leu⁷³-Gly¹⁷⁴ and Thr¹⁷⁹-Ile-Pro¹⁸¹ corresponding to the substrate subsites P2-distorted P1 and P1′-P2′-P3′, respectively, were identified as ‘hotspots’ that impaired inhibition when replaced with Ala (Betts & Anagli (2004) Biochemistry 43:2596-2604). Replacing the intact 27-residue peptide with the corresponding N- and C-terminal peptides, Asp¹⁶³-Gly¹⁷⁴ and Lys¹⁷⁵-Ala¹⁸⁹ (human sequence) , abolished calpain inhibition (Betts & Anagli (2004) supra). Taken together, these data indicate that DIII anchoring by region B enhances inhibitory activity >10-fold and support the structure-based occluding-loop mechanism. The Ca²⁺-dependent reversible interaction between calpastatin and calpain is biologically relevant and, in light of the length of the occluding loop, it indicates that the wild-type inhibitor, unlike the shorter loop mutants, may recycle once dissociated from the protease. This overcomes the need for new synthesis of calpastatin to maintain inhibition under conditions of fluctuating Ca²⁺ levels.

Only regions A, C and the DI-II-binding residues of B are conserved among calpastatins (FIG. 5). The divergent intervening sequences connecting regions A, B and C are devoid of electron density. NMR analysis of the complex between ¹⁵N-labelled calpastatin and unlabelled calpain corroborated that the intervening sequences of calpastatin are intrinsically disordered (FIG. 5). Conversely, calpastatin regions A, B and C bind calpain, become ordered and tumble slowly in solution as part of the 111 kDa complex, and were undetectable by NMR. DIII contains hotspots for proteolysis in the free and Ca²⁺-bound m-calpain (Moldoveanu, et al. (2001) supra), and its extensive interaction with calpastatin in the complex supports its resistance to trypsin. The modular organization of calpastatin induced on binding to calpain emphasizes the role of the interspersed, flexible/disordered segments in the folding-binding transitions of the structured regions at distant sites in calpain. Similar disorder was confirmed by NMR for a complex between m-calpain and repeat 1 of calpastatin at 10 μM CaCl₂ (Kiss, et al. (2008) FEBS Lett. 582:2149-2154). Calpastatin regions A and C interact with calpain whereas the intervening regions are disordered (Kiss, et al. (2008) supra). Notably, calpastatin region B corresponding to Lys¹⁷⁶-Leu¹⁸⁸, which in the instant complex targets mainly DI, also interacts with calpain. At this CaCl₂ level the protease core is not aligned for catalysis and, therefore, the N-terminus of region B does not contact the unprimed side of the active site nor the DIII. The disorder in the calpain-bound calpastatin prompted the prediction of minimal global conformational changes in calpain instigated by calpastatin binding. This indicated that the rearrangement of calpain domains is mainly induced by Ca²⁺. Local calpastatin-induced conformational changes in calpain are predicted for the gating loops of the protease core, found in alternative conformations in the structure of μI-II free or bound to inhibitors (Moldoveanu, et al. (2002) supra; Moldoveanu, et al. (2004) supra).

The calpain-calpastatin structure offers an unprecedented opportunity to study the Ca²⁺-bound conformation of m-calpain. The structures of inactive calpain (Hosfield, et al. (1999) Reverter, et al. (2002) Biol. Chem. 383:1415-1422; Strobl, et al. (2000) Proc. Natl. Acad. Sci. USA 97:588-592; Pal, et al. (2003) Structure 11:1521-1526), the Ca²⁺-bound protease core (Moldoveanu, et al. (2002) supra; Davis, et al. (2007) J. Mol. Biol. 366:216-229; Moldoveanu, et al. (2003) Nature Struct. Biol. 10:371-378) and Ca²⁺-bound and free DVI (Blanchard, et al. (1997) Nature Struct. Biol. 4:532-538; Lin, et al. (1997) Nature Struct. Biol. 4:539-547) have generated valuable, incomplete models for calpain activation (Suzuki, et al. (2004) supra). The calpain-calpastatin structure disclosed herein represents the Ca²⁺-activated conformation of m-calpain revealed by the realignment for catalysis of the protease core DI-II by two Ca²⁺ atoms, as described for μI-II (Moldoveanu, et al. (2002) supra)(Table 5). DI-II is intimately associated with DIII, which undergoes conformational changes to interact specifically with DI, serving to stabilize the protease core and to maximize its catalytic activity. The EF-hand DIV and DVI bind four Ca²⁺ atoms each (Dutt, et al. (2000) Biochem. J. 348:37-43), mediate the heterodimer interface by pairing of EF-hand 5 as in the apo-calpain (Hosfield, et al. (1999) supra) , and show small changes between the Ca²⁺-bound and free conformation (Table 5). Of significance is the Ca²⁺-induced displacement from DVI of the N-terminal anchor peptide (Hosfield, et al. (1999) supra), which is unstructured in the complex.

TABLE 5

| Coordinating Residue | Ca²⁺ Coordinations |
|---|---|
| | Å to Ca²⁺ 101 |
| Asp⁹⁶ OD2 | 2.2 |
| Gly⁹¹ O | 2.4 |
| Asp⁹⁶ OD1 | 2.5 |
| Glu¹⁷⁵ OE2 | 2.6 |
| Glu¹⁷⁵ OE1 | 2.8 |
| Ile⁸⁹ O | 2.9 |
| | Å to Ca²⁺ 201 |
| Glu²⁹² OE1 | 2.4 |
| Glu²⁹² OE2 | 2.5 |
| Glu³²³ O | 2.6 |
| Asp²⁹⁹ OD2 | 2.7 |
| Gln³¹⁹ O | 2.9 |
| Asp³²¹ OD1 | 3.3 |
| | Å to Ca²⁺ 401 |
| Glu⁵⁴⁷ O | 2.2 |
| Glu⁵⁵² OE1 | 2.5 |
| Asp⁵⁴⁵ OD1 | 2.6 |
| Asp⁵⁴⁵ OD2 | 3.0 |
| Ala⁵⁴² O | 3.6 |
| Asp⁵⁴⁵ O | 3.6 |
| | Å to Ca²⁺ 402 |
| Glu⁵⁹⁶ OE2 | 2.1 |
| Ser⁵⁸⁹ OG | 2.4 |
| Asp⁵⁸⁷ OD2 | 2.6 |
| Glu⁵⁹⁶ OE1 | 2.7 |
| Glu⁵⁴⁷ OE2 | 2.7 |
| Asp⁵⁸⁵ OD1 | 2.8 |
| Lys⁵⁹¹ O | 2.8 |
| | Å to Ca²⁺ 403 |
| Thr⁶²¹ O | 2.2 |
| Asp⁶¹⁵ OD1 | 2.3 |
| Glu⁶²⁶ OE1 | 2.3 |
| Asp⁶¹⁷ OD1 | 2.6 |
| Ser⁶¹⁹ OG | 2.7 |
| | Å to Ca²⁺ 404 |
| Asp⁶⁵⁸ OD2 | 2.3 |
| Asp⁶⁵⁸ OD1 | 2.3 |
| Asp⁶⁶⁰ OD1 | 2.5 |
| Asp⁶⁶¹ OD1 | 2.5 |
| Asp⁶⁶⁰ OD2 | 2.6 |
| | Å to Ca²⁺ 601 |
| Glu¹²¹ OE2 | 2.2 |
| Glu¹¹⁶ O | 2.4 |
| Asp¹¹⁴ OD1 | 2.4 |
| Ala¹¹¹ O | 2.9 |
| Asp¹¹⁴ OD2 | 3.0 |
| Glu¹²¹ OE1 | 3.3 |
| | Å to Ca²⁺ 602 |
| Asp¹⁵⁶ OD1 | 2.2 |
| Lys¹⁶⁰ O | 2.4 |
| Glu¹⁶⁵ OE1 | 2.4 |
| Asp¹⁵⁶ OD2 | 2.5 |
| Glu¹¹⁶ OE2 | 2.8 |
| Glu¹⁶⁵ OE2 | 2.9 |
| Thr¹⁵⁸ OG1 | 3.0 |
| | Å to Ca²⁺ 603 |
| Asp¹⁸⁶ OD1 | 2.2 |
| Glu¹⁹⁵ OE1 | 2.3 |
| Asp¹⁸⁴ OD2 | 2.5 |
| Ser¹⁸⁸ OG | 2.6 |
| Glu¹⁹⁵ OE2 | 2.9 |
| Thr¹⁹⁰ O | 3.0 |
| | Å to Ca²⁺ 604 |
| Asn²³⁰ OD1 | 2.0 |
| Asp²²⁷ OD1 | 2.3 |
| Asp²²⁹ OD2 | 2.4 |
| Asp¹³⁹ OD2 | 2.5 |
| Asp²²⁹ OD1 | 2.6 |
| Asp¹³⁹ OD1 | 2.9 |

To investigate the Ca²⁺-induced conformational changes leading to the heterodimer activation, the Ca²⁺-bound and free structure (Reverter, et al. (2002) supra) was aligned by overlapping DIII, which provides a central scaffold for the (re)arrangements of the vicinal domains through protein-protein interactions. On binding Ca²⁺, the upper DI-II lobe moves dorsal to frontal with respect to DIII, whereas the lower DIV-DVI lobe moves in the opposite direction. The tension on either side of the protease core, postulated in light of the Ca²⁺-free m-calpain structure (Hosfield, et al. (1999) supra) and confirmed extensively biochemically (Suzuki, et al. (2004) supra), is overcome by Ca²⁺ binding. The discovery of the active conformation of the DI-III ensemble is significant as it identifies the missing conserved features at the extensive DI-II-DIII interface (FIG. 6).

The importance of this interface was extrapolated from structural analysis of the isolated protease core DI-II of m-calpain, μI-II. Owing to intrinsic instability of residues Gly¹⁹⁷-Gly²¹⁰ in DI, the unprimed side of the active site in μI-II collapses, diminishing activity >1,000-fold compared to full-length m-calpain (Moldoveanu, et al. (2003) supra). In the Ca²⁺-bound heterodimer, residues 197-210 are stabilized, through salt bridges and hydrogen bonds, by conserved basic residues in DIII (FIG. 6). In particular, Arg⁴¹⁷ and Arg⁴²⁰ from the basic loop, which adopts a different conformation in Ca²⁺-free calpain, and Arg⁴⁶⁹ and Arg⁵″ from the β-sandwich core of DIII may provide critical support for the labile active site (FIG. 6).

In limb-girdle muscular dystrophy (LGMD)-2A patients, p94 (calpain 3) point mutants in DIII result in the typical atrophic phenotypes associated with impaired p94 activity in limb-girdle and trunk muscles (Kramerova, et al. (2007) Biochim. Biophys. Acta 1772:128-144). The positions 490, 493, 541 and 572 in p94, corresponding to the conserved Arg residues (417, 420, 469 and 500, respectively) in DIII of m-calpain, are mutated to Trp, Gln or Pro in both familial and sporadic forms of the disease (Jia, et al. (2001) Biophys. J. 80:2590-2596). The DI-II-DIII active interface was probed by mutagenesis in m-calpain. The R417A and R420A substitutions decreased m-calpain activity to one-half and one-quarter, respectively, and doubled the Ca²⁺ requirement in the latter. The R469A decreased activity >60-fold and doubled the Ca^{2 +} requirement. Conservative Lys substitutions at 417, 420 or 469 did not rescue the phenotypes of Ala mutations, collectively underscoring the importance of this interface for sustaining maximal calpain activity and providing an explanation for the effect of disease-causing p94 substitutions in LGMD-2A patients.

Calpastatin inhibits m- and μ-calpains (Wendt, et al. (2004) supra), which share the 28 kDa regulatory subunit predicted in both to bind calpastatin region C similarly (FIG. 2B). Modeling of the complex between μ-calpain and calpastatin illustrated that calpastatin regions A and B bind conserved pockets of the 80 kDa catalytic subunit and probably produce a similar set of inhibitory interactions with μ-calpain. It has been reported that μI-II is not inhibited by calpastatin repeat (Moldoveanu, et al. (2002) supra). Herein it is shown that trimming down calpastatin repeat 1 from either end to the 18-residue peptide B1 (Ala¹⁷²-Glu¹⁶⁹) increased inhibitory potency for μI-II. The 86-residue repeat 1 (134-219) reduced μI-II activity with an IC₅₀ of 154.7±16.3 μM, whereas the 57-residue regions BC (Asp¹⁶³-Thr²¹⁹) and AB (Ala¹⁴⁴-Lys¹⁹⁰) and the peptide B1 inhibited μI-II with IC₅₀ values of 121.8±24.4, 80.5±10.2 and 24.5±5.3 μM, respectively (mean ±s.e.m.). In contrast, repeat 1 and fragments AB and BC exhibited low nanomolar IC₅₀ values toward m-calpain. These results confirm the specificity of calpastatin for the heterodimeric m- and μ-calpains and indicate that calpains that lack the small subunit, DIV and/or DIII may not support the same set of inhibitory interactions with calpastatin (Suzuki, et al. (2004) supra).

The calpain and calpastatin proteins represent a major ubiquitous cellular proteolytic system, the imbalance of which has been implicated in necrosis associated with stroke and neuronal injury and perhaps Alzheimer's disease, heart disease, cataract formation, type 2 diabetes, cancer and LGMD-2A (Saez, et al. (2006) Drug Discov. Today 11:917-923). The instant study shows the mechanisms of activation by Ca²⁺ and inhibition by calpastatin of m- and μ-calpains (FIG. 7). Additional mechanisms of regulation for the calpain-calpastatin system include phosphorylation, membrane targeting and differential localization. The details of calpastatin specificity for the heterodimers can now be used in the design of new therapeutic agents.

1. A method for facilitating the tenderization of meat comprising contacting a meat product with an effective amount of a stabilized variant calpastatin comprising an occluding loop of inhibitor region B so that tenderization of the meat product is facilitated.

2. The method of claim 1, wherein the stabilized variant calpastatin comprises an insertion or deletion in the occluding loop of inhibitor region B.

3. The method of claim 1, wherein the stabilized variant calpastatin is truncated.

4. The method of claim 1, further comprising domains A, B and C of inhibitory repeat 1, repeat 2, repeat 3, or repeat 4.

5. The method of claim 4, wherein sequences between domain A and B or B and C of the stabilized variant calpastatin have been modified for enhanced protease resistance.

6. The method of claim 1, wherein the sequence of the occluding loop of inhibitor region B of the stabilized variant calpastatin is Gly-Ile-Lys-Glu-Gly (SEQ ID NO:2), Gly-Lys-Arg-Glu-Val (SEQ ID NO:3), Gly-Glu-Lys-Glu-Glu (SEQ ID NO:4), Gly-Lys-Arg-Glu-Ser (SEQ ID NO:5), Gly-Glu-Arg-Asp-Asp (SEQ ID NO:6), or Gly-Lys-Arg-Glu-Gly (SEQ ID NO:7).

7. The method of claim 1, wherein the stabilized variant calpastatin is a fusion protein.

8. The method of claim 7, wherein the fusion protein comprises calpain.

9. A method for facilitating muscle growth comprising contacting muscle tissue with an effective amount of a destabilized variant calpastatin comprising an occluding loop of inhibitor region B so muscle growth is facilitated.

10. The method of claim 9, wherein the destabilized variant calpastatin comprises an insertion or deletion in the occluding loop of inhibitor region B.

11. The method of claim 9, wherein the destabilized variant calpastatin is truncated.

12. The method of claim 9, wherein the destabilized variant calpastatin further comprises domains A, B and C of inhibitory repeat 1, repeat 2, repeat 3, or repeat 4.

13. The method of claim 12, wherein sequences between domain A and B or B and C of the destabilized variant calpastatin have been modified for enhanced protease sensitivity.

14. The method of claim 9, wherein the sequence of the occluding loop of inhibitor region B of the destabilized variant calpastatin is Gly-Ile-Lys-Glu-Gly (SEQ ID NO:2), Gly-Lys-Arg-Glu-Val (SEQ ID NO:3), Gly-Glu-Lys-Glu-Glu (SEQ ID NO:4), Gly-Lys-Arg-Glu-Ser (SEQ ID NO:5), Gly-Glu-Arg-Asp-Asp (SEQ ID NO:6), or Gly-Lys-Arg-Glu-Gly (SEQ ID NO:7).

15. An isolated variant calpastatin comprising an occluding loop of inhibitor region B;
domains A, B and C of inhibitory repeat 1, repeat 2, repeat 3, or repeat 4; and sequences between domains A, B and C, wherein the occluding loop of inhibitor region B has an insertion or deletion and one or more of the sequences between domains A, B and C have been modified for enhanced protease resistance or enhanced protease sensitivity.

16. The variant calpastatin of claim 15, wherein the calpastatin is truncated.

17-18. (canceled)

19. The variant calpastatin of claim 15, wherein the sequence of the occluding loop of inhibitor region B is Gly-Ile-Lys-Glu-Gly (SEQ ID NO:2), Gly-Lys-Arg-Glu-Val (SEQ ID NO:3), Gly-Glu-Lys-Glu-Glu (SEQ ID NO:4), Gly-Lys-Arg-Glu-Ser (SEQ ID NO:5), Gly-Glu-Arg-Asp-Asp (SEQ ID NO:6), or Gly-Lys-Arg-Glu-Gly (SEQ ID NO:7).

20. The variant calpastatin of claim 15, wherein the variant calpastatin is a fusion protein.

21. The variant calpastatin of claim 20, wherein said fusion protein comprises calpain.

22. An isolated nucleic acid molecule encoding a variant calpastatin of claim 15.

23. An isolated vector comprising the nucleic acid molecule of claim 22.

24. An isolated host cell comprising the vector of claim 23.

25. A non-human transgenic animal that expresses a variant calpastatin of claim 15.

26. A method for activating/inactivating or stabilizing/destabilizing calpain comprising contacting calpain with an effective amount of a variant calpastatin of claim 15 so that the calpain is activated/inactivated or stabilized/destabilized.

27. An isolated variant calpain with enhanced stability compared to wild-type calpain.

28. The variant calpain of claim 27, wherein the calpain has been modified for enhanced protease resistance.

29. The variant calpain of claim 27 or 28, wherein the variant calpain is a fusion protein.

30. The variant calpain of claim 27, 28 or 29, wherein said fusion protein comprises calpastatin.

31. An isolated nucleic acid molecule encoding a variant calpain of claim 27.

32. An isolated vector comprising the nucleic acid molecule of claim 31.

33. An isolated host cell comprising the vector of claim 32.

34. A non-human transgenic animal that expresses a variant calpain of claim 27.


SEQUENCE LISTING (22 sequences)

SEQ ID NO: 1 - protein, 120 aa, Rattus norvegicus
MESTLNKLSDKSGVNAALDDLIDTLGECEDTNKDDPPYTGPVVLDPMDSTYLEALGIKEGTIPPEYRKLLEKNEAITGPLPDSPKPMGIDHAIDALSSDFTCSSPTGKQTEKEKSTGESS

SEQ ID NO: 2 - protein, 5 aa, Rattus norvegicus
GIKEG

SEQ ID NO: 3 - protein, 5 aa, Homo sapiens
GKREV

SEQ ID NO: 4 - protein, 5 aa, Sus scrofa
GEKEE

SEQ ID NO: 5 - protein, 5 aa, Bos taurus
GKRES

SEQ ID NO: 6 - protein, 5 aa, Ovis aries
GERDD

SEQ ID NO: 7 - protein, 5 aa, Gallus gallus
GKREG

SEQ ID NO: 8 - DNA, 33 bp, Artificial sequence
  Synthetic oligonucleotide
gcatggccatggacaagtcaggcgtgaatgctg

SEQ ID NO: 9 - DNA, 37 bp, Artificial sequence
  Synthetic oligonucleotide
gtggtgctcgagttactttccagttggagagctacag

SEQ ID NO: 10 - DNA, 33 bp, Artificial sequence
  Synthetic oligonucleotide
gcatggccatggctgctttggatgacctgatag

SEQ ID NO: 11 - DNA, 37 bp, Artificial sequence
  Synthetic oligonucleotide
gtggtgctcgagttaggtgaaatcagatgaccaggca

SEQ ID NO: 12 - DNA, 32 bp, Artificial sequence
  Synthetic oligonucleotide
gcatggccatggacccaatggattctacctac

SEQ ID NO: 13 - DNA, 39 bp, Artificial sequence
  Synthetic oligonucleotide
gtggtgctcgagttaacaggtgaaatcagatgacaaggc

SEQ ID NO: 14 - DNA, 33 bp, Artificial sequence
  Synthetic oligonucleotide
gaaacttctggagaaataagaagctatcacagg

SEQ ID NO: 15 - DNA, 27 bp, Artificial sequence
  Synthetic oligonucleotide
ccagaagcatcgggcgcggcagaggaa

SEQ ID NO: 16 - DNA, 26 bp, Artificial sequence
  Synthetic oligonucleotide
ggcggcggcaggcgaagatgggtgag

SEQ ID NO: 17 - DNA, 29 bp, Artificial sequence
  Synthetic oligonucleotide
ccttcatcaacctcgcggaggtcctcaac

SEQ ID NO: 18 - DNA, 27 bp, Artificial sequence
  Synthetic oligonucleotide
ggcactgggtatagaagggactattcc

SEQ ID NO: 19 - DNA, 28 bp, Artificial sequence
  Synthetic oligonucleotide
gcactgggtataaaagggactattcctc

SEQ ID NO: 20 - DNA, 26 bp, Artificial sequence
  Synthetic oligonucleotide
ggcactgggtatagggactattcctc

SEQ ID NO: 21 - protein, 18 aa, Artificial sequence
  Synthetic oligonucleotide
ALGIKEGTIPPEYRKLLE

SEQ ID NO: 22 - protein, 4 aa, Rattus norvegicus
PPEY